(19) United States
(12) Patent Application Publication
(10) Pub. No.: US 2025/0250324 A1
(43) Pub. Date: Aug. 7, 2025

(54) NOVEL FUSION PROTEIN FOR ELIMINATING NEURODEGENERATIVE DISEASE-CAUSING FACTOR AND USE THEREOF

(71) Applicant: KOREA INSTITUTE OF SCIENCE AND TECHNOLOGY, Seoul (KR)

(72) Inventors: Sang Chul SHIN, Seoul (KR); Jinyoung PARK, Seoul (KR); Laon KIM, Yongin-si (KR); Sang Soo CHOI, Seoul (KR); Jinju SEO, Seoul (KR); Nak-Kyoon KIM, Seoul (KR); Hoon RYU, Seoul (KR)

(73) Assignee: KOREA INSTITUTE OF SCIENCE AND TECHNOLOGY, Seoul (KR)

(21) Appl. No.: 18/650,859

(22) Filed: Apr. 30, 2024

(30) Foreign Application Priority Data
Feb. 5, 2024 (KR) 10-2024-0017700

(51) Int. Cl. C07K 14/765; A61K 38/00; A61P 25/28

(52) U.S. Cl. CPC C07K 14/765; A61P 25/28; A61K 38/00; C07K 2319/09; C07K 2319/10

(57) ABSTRACT

A fusion protein of the present disclosure may induce the degradation of neurodegenerative disease-causing factors by binding to Tau and amyloid-beta proteins as the neurodegenerative disease-causing factors and activating autophagy. In addition, the fusion protein of the present disclosure penetrates a blood-brain barrier and is introduced into cells to be effectively delivered into nerve cells without a separate carrier, and has high stability and thus is expected to be used as a platform for the treatment of neurodegenerative diseases such as dementia and Alzheimer's disease.

### CROSS-REFERENCE TO RELATED APPLICATION

This application claims the benefit of Korean Patent Application No. 10-2024-0017700 filed on Feb. 5, 2024, in the Korean Intellectual Property Office, the entire disclosure of which is incorporated herein by reference for all purposes.

### REFERENCE TO AN ELECTRONIC SEQUENCE LISTING

The contents of the electronic sequence listing (“NewApp_0210520004_Sequence.xml”; Size is 44 kilobytes and it was created on Apr. 30, 2024) is herein incorporated by reference in its entirety.

### BACKGROUND

### 1. Field of the Invention

The present disclosure relates to a novel fusion protein for specifically eliminating Tau and amyloid beta (amyloid P), which are neurodegenerative disease-causing factors, and a use thereof.

### 2. Description of the Related Art

Human serum albumin (HSA) is widely distributed throughout the body, especially in the interstitium and blood compartment, and is involved in maintaining osmotic pressure. Albumin has a characteristic of increasing the half-life in vivo by binding to lipids and bilirubin, and it is known that the characteristic of albumin may be used to produce a recombinant fusion protein for treatment (Korean Patent Application Publication No. 10-2015-0058454). The fusion protein using albumin has an increased half-life in vivo to exhibit a lower frequency of injection and a higher level of therapeutic effect.

According to recently reported literature, it has been proven that albumin may penetrate a blood-brain barrier (BBB) by binding to a compound called photosensitizer (ZnPcS), and thus, research has been conducted to use albumin as a method for delivering therapeutic substances for brain diseases and brain cancer to the brain without physical treatment.

Dementia, a representative neurodegenerative disease, is divided into Alzheimer's disease, Lewy body dementia, frontotemporal dementia, etc., and has cognitive impairment as a representative symptom. According to the results of “Dementia Prevalence Survey” in Korea on Apr. 13, 2009, in normal elderly people over 65 years old, the annual incidence of dementia was approximately 1 to 3%, while in elderly people with mild cognitive impairment, the annual incidence of dementia was approximately 12 to 16%.

Representative factors that cause dementia include aggregation of Tau protein and amyloid beta. The Tau protein binds to microtubules and maintains the transport of substances within neurons, which are nerve cells. When hyperphosphorylation of the Tau protein occurs, the Tau protein falls off from the microtubules, and the fallen Tau protein forms Tau tangles, which are aggregates, and are deposited in brain tissue to cause Alzheimer's disease. In addition, when structural deformation occurs in the amyloid-beta protein, amyloid-beta forms a fibrous shape and accumulates excessively in a certain brain region to cause the symptoms, but particularly, amyloid aggregates that occur early have a very close effect on nervous system toxicity and the development of dementia. Aduhelm, a dementia disease therapeutic agent approved by the Food and Drug Administration (FDA), and Leqembi, which is in phase 3 clinical trials, bind to aggregated amyloid-beta and serve to disperse aggregated amyloid to alleviate the symptoms of dementia. The aduhelm improves cognitive ability as a mechanism for preventing the aggregation of amyloid-beta, but safety and effectiveness issues have been raised due to inflammation and other side effects, and new dementia therapeutic drugs are needed due to the high cost.

Intracellular protein degradation occurs through two mechanisms by lysosome and proteasome, but approximately 80% of cellular proteins are labeled with ubiquitin and then degraded in the cytoplasm and nucleus by the proteasome, which is called a ubiquitin-proteasome system (UPS). Autophagy, another protein degradation system, is a mechanism that degrades cytoplasmic contents to maintain homeostasis and is associated with various diseases such as infection and neurodegeneration. The activity of autophagy decreases with age, and many results show that increased autophagy exhibits a therapeutic effect on dementia. Several companies are attempting to develop therapeutic agents through strategies that enhance autophagy in disease conditions or degrade specific cargo by using autophagy.

Accordingly, the present inventors confirmed that as a platform for eliminating Tau and/or amyloid-beta as a neurodegenerative disease-causing factor, a novel fusion protein was prepared by binding to Tau and/or amyloid-beta protein and activates autophagy to eliminate an aggregated target protein and considering the convenience of administration and the possibility of action in the brain to penetrate the blood-brain barrier (BBB), and the fusion protein bound to the Tau protein to cause autophagy, thereby reducing the amount of Tau protein, and then completed the present disclosure.

### SUMMARY

Embodiments provide a novel fusion protein capable of inducing the degradation of neurodegenerative disease-causing factors in cells through an autophagy mechanism by passing through a BBB and penetrating a cell membrane without a separate carrier and a use for preventing or treating neurodegenerative diseases thereof.

However, technical aspects of the present disclosure are not limited to the aforementioned purpose and other aspects which are not mentioned may be clearly understood to those skilled in the art from the following description.

In the specification, amino acid sequences are listed in order from the N-terminus to the C-terminus.

A Tau protein and an amyloid-beta protein are misfolded due to unknown reasons to form non-specific aggregates, and at this time, it is known that the aggregated Tau or amyloid-beta protein is accumulated in the cells to exhibit neuronal toxicity and cause degenerative brain diseases such as dementia. Meanwhile, the Tau or amyloid-beta aggregates have a characteristic of not being soluble or degraded, and the present disclosure provides a novel fusion protein that induces the degradation of the aggregates.

According to an aspect, there is provided a fusion protein capable of inducing the degradation of a neurodegenerative disease-causing factor to eliminate the factor, in which the fusion protein has compositions of (1) to (4) below, and the compositions (1) to (4) above are linked by covalent bonds in order from the N-terminus to the C-terminus.
(1) Cell penetrating peptide (CPP)
(2) Human serum albumin (HSA)
(3) Neurodegenerative disease-causing factor binding peptide
(4) Autophagy inducing peptide (AIP)

As used in the present disclosure, the term “neurodegenerative disease-causing factor” refers to the above-described Tau protein and amyloid-beta protein and is interpreted to include aggregates of the Tau or amyloid-beta protein and variants thereof.

The neurodegenerative disease-causing factor peptide of the present disclosure may be a Tau binding peptide Tau_B or amyloid-beta binding peptide αβ_B, and the Tau_B may contain or consist of an amino acid sequence represented by SEQ ID NO: 7, and the αβ_B may include or consist of an amino acid sequence represented by SEQ ID NO: 8.

Meanwhile, the neurodegenerative disease-causing factor binding peptide of the present disclosure may further include a linker to the N-terminus and/or C-terminus and be linked to the HSA and/or AIP with a linker. In the present disclosure, the linker may consist of 1 to 10 a.a. as long as the linker does not affect the function of each domain, and in the specific experiment of the present disclosure, a linker consisting of an amino acid sequence represented by SEQ ID NO: 40 was used.

Meanwhile, the present inventors designed the fusion protein of the present disclosure to include a cell-penetrating peptide so as to penetrate into cells and bind to a target molecule, that is, a neurodegenerative disease-causing factor. As the cell-penetrating peptide, in Table 1 below, three types of peptides VAL, THR, and LRK were prepared, and it was confirmed that the cell-penetrating peptides may introduce the fusion protein of the present disclosure into cells. Accordingly, the cell-penetrating peptide of the present disclosure may include or consist of one or more amino acid sequences selected from the group consisting of SEQ ID NOs: 1 to 3.

In addition, the fusion protein of the present disclosure used human serum albumin (HSA) (expasy: P02768) to increase the in vivo half-life and penetrate the BBB. Specifically, the fusion protein of the present disclosure contains a HAS-derived peptide, and the HAS-derived peptide consists of amino acids in a region from amino acid at position 26 to amino acid at position 609 of wild-type HSA (SEQ ID NO: 6). In this specification, the HSA-derived peptide as a component included in the fusion protein of the present disclosure is represented as HSA, and unless specifically intended to distinguish between albumin and the albumin-derived peptide, the albumin-derived peptide may be indicated simply as albumin. Meanwhile, as the results of confirming through experiments whether the fusion protein binds to the Tau protein depending on the presence or absence of HSA and Tau_B, the present inventors confirmed that HAS exhibited the effect of increasing the binding of the fusion protein to the Tau protein, in addition to the improvement of in vivo safety and the function of the BBB penetration.

In this specification, the “autophagy inducing peptide (AIP)” is a peptide that activates autophagy, one of systems that degrade proteins in cells. The autophagy-activating peptide increases the expression of autophagy-activating proteins p62 and LC3, and the increased expression of p62 and LC3 activates autophagy to form autophagosomes targeting the autophagy-activating peptide to induce the degradation of the peptide. In other words, the autophagy-activating peptide serves to activate the autophagy mechanism and simultaneously label proteins to be degraded within the cells.

The present inventors developed PCM1 and D11 as autophagy-activating peptides, and experimentally confirmed that fusion proteins containing each autophagy-activating peptide induce binding to target molecules in the cells and the degradation thereof. In the present disclosure, the PCM1 may include or consist of an amino acid sequence represented by SEQ ID NO: 9, and the D11 may include or consist of an amino acid sequence represented by SEQ ID NO: 10.

Meanwhile, in the fusion protein of the present disclosure, CPP and HSA may be linked by a nuclear localization sequence or signal (NLS) peptide. The NLS transports specific substances (e.g., proteins) into the cell nucleus through nuclear pores and the nuclear localization sequence is not required for neurodegenerative disease-causing eliminating fusion protein activity in eukaryotes, but it is considered to enhance the activity of the system, particularly, to target the nucleic acid molecules within the nucleus by the sequence. In the present disclosure, the NLS may include or consist of an amino acid sequence represented by SEQ ID NO: 5.

The amino terminus of the fusion protein of the present disclosure may be bound with a protecting group, such as an acetyl group, a fluorenyl methoxycarbonyl group, a formyl group, a palmitoyl group, a myristyl group, a stearyl group, and polyethylene glycol (PEG), and the carboxy terminus of the fusion protein may be modified with a hydroxyl group (—OH), an amino group (—NH₂), an azide (—NHNH₂), or the like. In addition, the terminus of the peptide of the present disclosure or an R-residue (R-group) of the amino acid may be bound with fatty acids, oligosaccharides chains, all nanoparticles (gold particles, liposomes, heparin, hydrogel, etc.), amino acids, carrier proteins, and the like. The modifications of the amino acids described above serve to improve the potency and stability of the fusion protein of the present disclosure. As used in the present disclosure, the term “stability” refers to not only in vivo stability, but also storage stability (including storage stability at room temperature, refrigeration, and frozen storage).

Meanwhile, each peptide constituting the fusion protein of the present disclosure may be substituted, modified, or deleted with one or more amino acid sequences within the range of maintaining its function.

According to another aspect, there is provided a polynucleotide encoding the above-described fusion protein, an expression vector for the fusion protein containing the polynucleotide, and a cell transformed with the expression vector.

As used in the present disclosure, the term “polynucleotide” refers to a polymer of deoxyribonucleotides or ribonucleotides that exist in a single-stranded or double-stranded form. The polynucleotide encompasses RNA genome sequences, DNA (gDNA and cDNA), and RNA sequences transcribed therefrom, and includes analogs of a natural polynucleotide unless specifically stated otherwise.

In the present disclosure, the nucleotide sequence encoding the fusion protein includes not only a nucleotide sequence encoding the amino acid indicated by each sequence number, but also a nucleotide sequence having 80% or more, specifically 90% or more, more specifically 95% or more, much more specifically 98% or more, and most specifically 99% or more homology with the sequence, and includes any nucleotide sequence encoding a protein that shows an effect substantially identical or corresponding to the protein without limitation. In addition, if the sequence having the homology to the sequence is an amino acid sequence having biological activity substantially identical or corresponding to a conjugate protein of the sequence number described above, it is obvious that amino acid sequences in which some sequences are deleted, modified, substituted, or added are also included in the scope of the present disclosure.

As used in the present disclosure, the term “homology” refers to the degree of similarity to a nucleotide sequence encoding a protein or an amino acid sequence constituting the protein, but if the homology is sufficiently high, the expression product and protein of the corresponding gene may have the same or similar activity. In addition, the homology may be expressed as a percentage based on the degree of matching a given amino acid or nucleotide sequence. In this specification, its homologous sequence having identical or similar activity to the given amino acid or nucleotide sequence is indicated as “% homology”. For example, the homology may be confirmed by comparing sequences by using a standard software for calculating parameters such as score, identity, and similarity, specifically BLAST 2.0, or hybridization experiments that have been used under defined stringent conditions, and appropriate hybridization conditions defined are within the scope of the art and may be determined by methods well known to those skilled in the art.

In addition, the polynucleotide encoding the fusion protein may be variously modified in coding regions within a range without changing the amino acid sequence of the protein expressed in the coding region in consideration of a codon preferred in an organism to express the protein due to codon degeneracy. Accordingly, the polynucleotide may be included without limitation as long as it is a polynucleotide sequence encoding each protein.

In addition, the polynucleotide includes not only a nucleotide sequence encoding the amino acid sequence of the fusion protein, but also a complementary sequence to the sequence. The complementary sequences include not only a perfectly complementary sequence, but also a substantially complementary sequence, which may be, for example, sequences that may hybridize with the nucleotide sequence of the nucleotide sequence encoding the amino acid sequence of the fusion protein, under stringent conditions known in the art.

The hybridization requires two polynucleotides to have a complementary sequence even if mismatch between bases is possible according to the stringent degree of hybridization. The term “complementary” is used to describe a relation between bases of nucleotides which may be hybridized with each other. For example, for DNA, adenosine is complementary to thymine and cytosine is complementary to guanine. Accordingly, the present disclosure may include isolated polynucleotide fragments complementary to the overall sequence as well as a substantially similar polynucleotide sequence.

As used in the present disclosure, the term “expression vector” is a recombinant vector which is introduced into a suitable host cell to express a target protein and refers to a gene construct including a required regulatory element which is operably linked so that a gene insert is expressed. As used herein, the term “operably linked” means that a nucleic acid sequence encoding a target protein is functionally linked to a nucleic acid expression regulatory sequence to perform a general function. The operable linkage to the recombinant vector may be manufactured using a gene recombination technique well-known in the art, and site-specific DNA cleavage and linkage may be facilitated using enzymes and the like which are generally known in the art.

The suitable expression vector of the present disclosure may include a signal sequence for membrane targeting or secretion in addition to expression control elements such as a promoter, a start codon, a stop codon, a polyadenylation signal, and an enhancer. The start codon and the stop codon are generally considered as a part of the nucleotide sequence encoding an immunogenic target protein, and need to exhibit actions in the subject when the gene construct is administered and needs to be in frame with the coding sequence. General promoters may be constitutive or inducible, and include lac, tac, T3, and T7 promoters in prokaryotic cells; and not only simian virus 40 (SV40), mouse mammary tumor virus (MMTV) promoter, human immunodeficiency virus (HIV), including a long terminal repeat (LTR) promoter of HIV, Moloney virus, cytomegalovirus (CMV), Epstein Barr virus (EBV) and Rouss sarcoma virus (RSV) promoters, but also a β-actin promoter, human hemoglobin, human muscle creatine, and a human metallothionein-derived promoter in eukaryotic cells, but are not limited thereto.

In addition, the expression vector may include a selective marker for selecting host cells containing the vector. The selective marker is used to select cells transformed with a vector and may be used with markers that impart selectable phenotypes such as drug resistance, auxotrophy, resistance to cytotoxic agents, or expression of surface proteins. In an environment treated with a selective agent, only cells expressing the selective marker survive, so that transformed cells may be selected. In addition, when the vector is a replicable expression vector, the vector may include a replication origin, which is a specific nucleic acid sequence from which replication is initiated.

As the recombinant expression vector for inserting foreign genes, various types of vectors such as plasmid, virus, and cosmid may be used. The type of recombinant vector is not particularly limited as long as the recombinant vector functions to express a desired gene and produce a desired protein in various host cells of prokaryotic cells and eukaryotic cells, but a vector capable of mass-producing a promoter exhibiting strong activity and a foreign protein in a form similar to a natural state while retaining a strong expression force may be used.

To express the protein of the present disclosure, a combination of various hosts and vectors may be used. Expression vectors suitable for eukaryotic hosts are not limited to pCMV, pFLAG, pMYC, pHA, etc., but may include expression control sequences derived from SV40, bovine papillomavirus, adenovirus, adeno-associated virus, cytomegalovirus, and retrovirus, and the like. Expression vectors that may be used in bacterial hosts are not limited thereto, and may include bacterial plasmids obtained from Escherichia coli, including pET21a, pET, pRSET, pBluescript, pGEX2T, pUC vector, col E1, pCR1, pBR322, pMB9, or derivatives thereof, plasmids with a wider host range, such as RP4; phage DNA, which may be exemplified as phage lambda derivatives such as λgt10, λgt11, and NM989, and other DNA phages such as M13 and filamentous single-stranded DNA phage. For yeast cells, 2° C. plasmids or derivatives thereof may be used, and for insect cells, pVL941, etc. may be used.

The cells, for example, eukaryotic cells may be cells of yeast, fungi, protozoa, plants, higher plants and insects, or amphibians, or mammalian cells such as CHO, HeLa, HEK293, and COS-1, and for example, may be cultured cells (in vitro), graft cells and primary cell cultures (in vitro and ex vivo), and in vivo cells, commonly used in the art, and also mammalian cells, including humans. In addition, the organisms may be yeast, fungi, protozoa, plants, higher plants and insects, amphibians, or mammals.

A complex containing the albumin protein may pass intracellularly or through the blood-brain barrier (BBB) without a carrier, and at this time, the albumin protein may be used as a carrier to be used as a complex with a specific compound and may be delivered specifically to specific cells, and thus, by using the complex, a cancer treatment effect for specific cells may be improved.

The fusion protein of the present disclosure may eliminate Tau or amyloid beta deposited in the cells by penetrating the cell membrane.

According to an aspect, there is provided a pharmaceutical composition for the prevention or treatment of neurodegenerative diseases caused by aggregation and deposition of Tau or amyloid beta.

As used in the present disclosure, the term “neurodegenerative disease” refers to a disease that causes various symptoms due to degenerative changes occurring in nerve cells of the central nervous system and a loss of intrinsic functions corresponding to an affected area thereof. The neurodegenerative diseases specifically include dementia, Alzheimer's disease, Lou Gehrig's disease, Lewy dementia, frontotemporal dementia, Parkinson's disease, tremor, proximal lateral sclerosis, chorea, multiple sclerosis, progressive supranuclear palsy, and Huntington's disease.

As used in the present disclosure, the term “treatment” refers to all actions that improve or beneficially change the symptoms of a neurodegenerative disease by administering the composition of the present disclosure.

As used in the present disclosure, the term “prevention” refers to all actions that inhibit or delay a neurodegenerative disease or the possibility of developing the disease by administering the composition of the present disclosure.

The pharmaceutical composition may include a pharmaceutically acceptable carrier. The “pharmaceutically acceptable carrier” may refer to a carrier or a diluent which does not inhibit biological activity and properties of a compound to be injected without stimulating organisms. Herein, the meaning of “pharmaceutically acceptable” means that the pharmaceutical composition does not inhibit the activity of the active ingredient and does not have more than adaptable toxicity of an application (prescription) target.

The type of carrier usable in the present disclosure may be used in any carrier which is commonly used in the art and pharmaceutically acceptable. Non-limitative examples of the carrier may include a saline solution, sterile water, a ringer solution, a buffer saline solution, an albumin injection solution, a dextrose solution, a maltodextrin solution, glycerol, ethanol, and the like. These carriers may be used alone or in combination of two or more kinds. The pharmaceutical composition may be prepared into an oral formulation or a parenteral formulation depending on a route of administration by a conventional method known in the art, including the pharmaceutically acceptable carrier in addition to the active ingredient.

The pharmaceutical composition may be formulated and used in the form of oral formulations such as powders, granules, tablets, capsules, suspensions, emulsions, syrups, aerosols, etc., external preparations, suppositories, and sterile injectable solutions according to a general method. When the pharmaceutical composition is formulated, the formulation may be prepared by adding diluents or excipients, such as a filler, an extender, a binder, a wetting agent, a disintegrating agent, or a surfactant, which are generally used.

When the pharmaceutical composition is prepared as an oral formulation, the pharmaceutical composition may be formulated into powders, granules, tablets, pills, sugar-coated tablets, capsules, solutions, gels, syrups, suspensions, wafers, etc. according to methods known in the art along with a suitable carrier. At this time, examples of suitable pharmaceutically acceptable carriers may include sugars such as lactose, glucose, sucrose, dextrose, sorbitol, mannitol, and xylitol, starches such as corn starch, potato starch, and wheat starch, celluloses such as cellulose, methylcellulose, ethylcellulose, sodium carboxymethylcellulose and hydroxypropylmethylcellulose, polyvinyl pyrrolidone, water, methylhydroxybenzoate, propylhydroxybenzoate, magnesium stearate, mineral oil, malt, gelatin, talc, polyol, vegetable oil, etc. When the pharmaceutical composition is formulated, the pharmaceutical composition may be formulated by including diluents and/or excipients, such as a filler, an extender, a binder, a wetting agent, a disintegrating agent, a surfactant, etc., if necessary.

When the pharmaceutical composition is prepared as a parenteral formulation, the pharmaceutical composition may be formulated in the form of injections, transdermal administration, nasal inhalation, and suppositories together with a suitable carrier according to methods known in the art. When formulated as an injection, suitable carriers may include sterile water, ethanol, polyols such as glycerol or propylene glycol, or mixtures thereof, desirably Ringer's solution, phosphate buffered saline (PBS) or sterile injectable solution containing triethanol amine, isotonic solutions such as 5% dextrose, or the like. When formulated as a transdermal agent, the pharmaceutical composition may be formulated in the form of ointments, creams, lotions, gels, external liquids, pastas, liniments, and aerosols. In the case of a nasal inhalant, the pharmaceutical composition may be formulated in the form of an aerosol spray using a suitable propellant such as dichlorofluoromethane, trichlorofluoromethane, dichlorotetrafluoroethane, carbon dioxide, etc. When formulated as a suppository, the bases may be used with witepsol, tween 61, polyethylene glycols, cacao fat, laurinum, polyoxyethylene sorbitan fatty acid esters, polyoxyethylene stearates, sorbitan fatty acid ester, etc.

The pharmaceutical composition may be administered in a pharmaceutically effective amount, and the term “pharmaceutically effective amount” means an amount enough to treat or prevent a disease at a reasonable benefit/risk ratio applicable to medical treatment or prevention. The effective dose level may be determined depending on factors including the severity of a disease, the activity of a drug, the age, body weight, health, and sex of a patient, the sensitivity to the drug of the patient, the administration time, administration route, excretion rate, and treatment period of the composition of the present disclosure used, combinations with the composition of the present disclosure used or simultaneously used drugs, and other factors well-known in a medical field. The pharmaceutical composition may be administered alone or in combination with ingredients known to exhibit therapeutic effects on known cancer diseases. Considering all of the factors, it is important to administer an amount capable of obtaining a maximum effect in a minimum amount without side effects.

The dose of the pharmaceutical composition may be determined by those skilled in the art by considering the purpose of use, the severity of a disease, the age, body weight, sex, and medical history of a patient, a type of material to be used as an effective ingredient, etc. For example, the pharmaceutical composition of the present disclosure may be administered in an amount of about 0.1 ng to about 1,000 mg/kg, desirably 1 ng to about 100 mg/kg per adult, and the frequency of administration of the composition of the present disclosure is specifically not limited thereto, but may be administered once a day, or administered several times by dividing the dose. The dose or frequency of administration does not limit the scope of the present disclosure in any aspect.

Further, the present disclosure provides a method for preventing or treating neurodegenerative diseases including administering the fusion protein of the present disclosure to a subject.

In the present disclosure, the “subject” includes, without limitation, mammals including rats, livestock, humans, birds, reptiles, farmed fish, etc., that develop or are at risk of developing neurodegenerative diseases.

The pharmaceutical composition may be administered singly or multiply in a pharmaceutically effective amount. At this time, the composition may be formulated and administered in the form of a solution, powder, aerosol, injection, infusion solution (Ringer's solution), capsule, pill, tablet, suppository, or patch. The route of administration of the pharmaceutical composition for preventing or treating cancer may be administered through any general route as long as the pharmaceutical composition may reach a target tissue.

The pharmaceutical composition is not particularly limited thereto, but may be administered through a route such as intraperitoneal, intravenous, intramuscular, subcutaneous, intradermal, transdermal patch, oral, intranasal, intrapulmonary, intrarectal administration, etc., depending on the purpose.

According to the embodiments, the fusion protein for eliminating neurodegenerative disease-causing factors, including the fusion protein of the present disclosure, binds to the Tau or amyloid-beta protein that causes Alzheimer's disease and activates autophagy to induce the degradation of Tau or amyloid-beta. In addition, the fusion protein of the present disclosure may pass through the blood-brain barrier and be introduced into cells to be effectively delivered into nerve cells even without a separate carrier and may be used as a platform for treating neurodegenerative diseases such as Alzheimer's disease and dementia by effectively eliminating intracellular deposition of Tau and amyloid-beta with high in vivo stability.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a schematic diagram of a fusion protein for eliminating neurodegenerative disease-causing factors designed in the present disclosure;

FIGS. 2A and 2B are schematic diagrams of structures of DNA vectors expressing fusion proteins for eliminating neurodegenerative disease-causing factors designed in the present disclosure;

FIGS. 3A to 3C are diagrams illustrating protein expression levels and yields to confirm the solubility of proteins expressed in E. coli transformed with an E. coli expression vector including fusion protein coding genes for eliminating neurodegenerative disease-causing factors designed in the present disclosure. “M” refers to a protein marker (kDa), “I” refers to induction of protein expression, “C” refers to a cell lysate after induction of protein expression, and “S” refers to a supernatant obtained by centrifugation after cell disruption, and “Ni—B” refers to a state in which the expressed fusion protein is bound to nickel beads. “P−” refers to before cleavage of 8 histidine tags using PreScission protease, and “P+” refers to after cleavage thereof,

FIGS. 4A and 4B are diagrams illustrating protein expression levels and yields to confirm the solubility of proteins expressed in E. coli transformed with an E. coli expression vector including fusion protein coding genes in which an albumin portion is omitted from the fusion proteins for eliminating neurodegenerative disease-causing factors designed in the present disclosure;

FIGS. 5A and 5B are diagrams illustrating results of pure separation and purification of the fusion proteins for eliminating the neurodegenerative disease-causing factors designed in the present disclosure using affinity chromatography and size exclusion chromatography using a nickel (Ni-NTA) column. “C” refers to a cell lysate after inducing protein expression, “S” refers to a supernatant obtained by centrifugation after cell disruption, “FT” refers to a flow through, and “4%” refers to a flow through after 20 mM imidazole washing, and “E” is a fraction according to an imidazole concentration gradient or each fraction separated and purified by protein exclusion chromatography;

FIGS. 6A and 6B are results of confirming through immunoprecipitation binding between proteins expressed after transfecting HEK293T cells with GFP-Tau plasmid and VAL-NLS-HSA-Tau_B-PCM1, VAL-NLS-Tau_B-PCM1, VAL-NLS-HSA-PCM1, MTD2-NLS-Tau_B-D11, and VAL-NLS-HSA-Tau_B-D11 plasmids, which are fusion proteins for eliminating neurodegenerative disease-causing factors designed in the present disclosure (FIG. 6A), and results of confirming through western blotting a Tau protein amount and protein amounts of autophagy markers p62 and LC3B after transfecting HeLa cells with the plasmids under the same conditions (FIG. 6B);

FIG. 7 is results of confirming through western blotting the protein amounts of autophagy markers Beclin1, p62, and LC3B by treating chloroquine (CQ) at a concentration of 25 μM for 24 hours after transfecting A549 cells with VAL-NLS-Tau_B-PCM1, VAL-NLS-HSA-Tau_B-PCM1, VAL-NLS-αβ_B-PCM1, and VAL-NLS-HSA-αβ_B-PCM1, which are protein proteins for eliminating neurodegenerative disease-causing factors designed in the present disclosure;

FIG. 8A is a result of confirming through immunoprecipitation binding between proteins expressed after transfecting HEK293T cells with GFP-Tau plasmid and VAL-NLS-HSA-PCM1, VAL-NLS-HSA-Tau_B-PCM1, and VAL-NLS-Tau_B-PCM1 plasmids.

FIG. 8B is a result of confirming through western blotting the Tau protein amount and the protein amounts of autophagy markers p62 and LC3B after transfecting HeLa cells with GFP-Tau plasmid and VAL-NLS-HSA-Tau_B-PCM1, and VAL-NLS-Tau_B-PCM1 plasmids.

FIG. 9A is a tau-overexpressed dementia model.

FIG. 9B is results of confirming the effects of the fusion proteins according to the present disclosure in the tau-overexpressed dementia model of FIG. 9A.

### DETAILED DESCRIPTION

The present disclosure may have various modifications and various Examples, and specific Examples will be hereinafter illustrated in the drawings and described in detail in the detailed description. However, the present disclosure is not limited to specific embodiments, and it should be understood that the present disclosure covers all the modifications, equivalents and replacements within the idea and technical scope of the present disclosure. In explaining the present disclosure, if it is judged that a detailed description of related known technology may obscure the gist of the present disclosure, the detailed description will be omitted.

### EXAMPLES

### Example 1. Construction of System for Eliminating Neurodegenerative Disease-Causing Factors

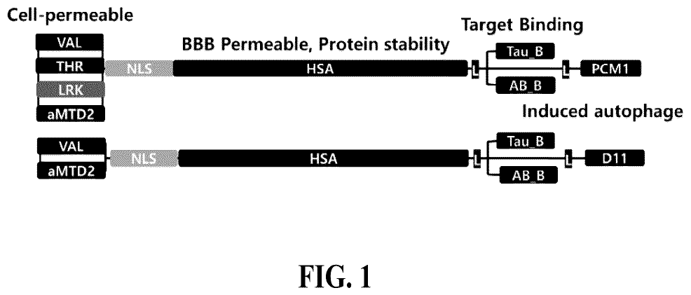

The present inventors constructed a fusion protein by specifically binding to aggregated Tau protein (Tau) or amyloid beta (αβ), which caused dementia, without a separate carrier to eliminate the protein for a system for eliminating neurodegenerative disease-causing factors. Specifically, the fusion protein of the present disclosure included 1) a protein portion (peptide) capable of specifically binding to Tau or amyloid-beta, 2) a protein portion (peptide) capable of activating autophagy that degraded the proteins within cells, 3) a cell penetrating peptide (CPP), and 4) an albumin protein. The specific structure of the fusion protein was illustrated in FIG. 1.

Hereinafter, a system was constructed by processes of specifically the type of fusion protein having functions of binding and eliminating a Tau protein as a disease-causing factor by confirming the binding of the fusion protein to the Tau protein and confirming an elimination pattern of the Tau protein by the fusion protein.

### 1-1. Selection of Cell Penetrating Peptide (CPP)

First, a peptide capable of improving cell penetration was selected to prepare a fusion protein capable of effectively moving into a cell where a target protein was located.

Specifically, based on the peptide with improved cell penetration efficiency reported in (US20180171322A1), three types of cell-penetrating peptides predicted to introduce the fusion protein of the present disclosure into cells were prepared and named VAL, THR, and LRK, respectively. As a control group, aMTD2 reported in US20180171322A1 was used.

An amino acid sequence of each CPP was shown in Table 1 below.

TABLE 1

| SEQ | | |
|---|---|---|
| ID | | |
| NO: | Name | Sequence (N-term → C-term) |
| 1 | VAL | VALLPAVP |
| 2 | THR | THRPPMWSPVWP |
| 3 | LRK | LRKLRKRLL |
| 4 | aMTD2 | VAALLPAVVVAP |

### 1-2. Selection of Nuclear Localization Sequence or Signal (NLS)

In addition, a nuclear localization sequence was included to transport the fusion protein of the present disclosure into the cell nucleus. Specifically, an NLS protein (human Sart3: expasy No; Q15020) was used with an NLS protein (SEQ ID NO: 5) having an amino acid sequence from arginine (Arg) at position 601 to asparagine (Asn) at position 649 for gene amplification.

### 1-3. Selection of Human Serum Albumin (HSA)

Next, a human serum albumin (HSA) protein was known to penetrate the blood-brain barrier (BBB). In addition, the HSA protein was used to increase the stability of the fusion protein of the present disclosure, enable the BBB penetration, and increase in vivo half-life. Specifically, a human albumin protein (human serum albumin: expasy No; P02768) used to prepare the fusion protein of the present disclosure was an HAS protein (SEQ ID NO: 6) having an amino acid sequence from aspartate (Asp) at position 26 to leucine (Lue) at position 609 for gene amplification.

### 1-4. Selection of Peptides for Targeting Dementia-Causing Factors

In order to specifically target aggregated Tau or amyloid-beta (αβ), the fusion protein of the present disclosure included a peptide binding specifically to Tau or amyloid-beta. The Tau binding peptide (Tau_B; SEQ ID NO: 7) and amyloid-beta binding peptide (αβ_B; SEQ ID NO: 8) were selected, respectively.

### 1-5. Selection of Autophagy Inducing Peptide (AIP)

Next, the fusion protein of the present disclosure was designed to include an autophagy inducing peptide so as to bind to a target and activate an autophagy to induce degradation of the target protein. The autophagy inducing peptide was used with PCM1 (SEQ ID NO: 9) or D11 (SEQ ID NO: 10) peptide.

There are two types of fusion proteins designed through the process: a fusion protein including CPP (VAL, THR, LRK, aMTD2), NLS, HAS, Tau_B, αβ_B, and PCM1 peptides, and a fusion protein including CPP (VAL, aMTD2), NLS, HAS, Tau_B, αβ_B, and D11 peptides, respectively (FIG. 1).

### Example 2. Preparation of Fusion Proteins

### 2-1. Preparation of Fusion Protein Expression Vectors

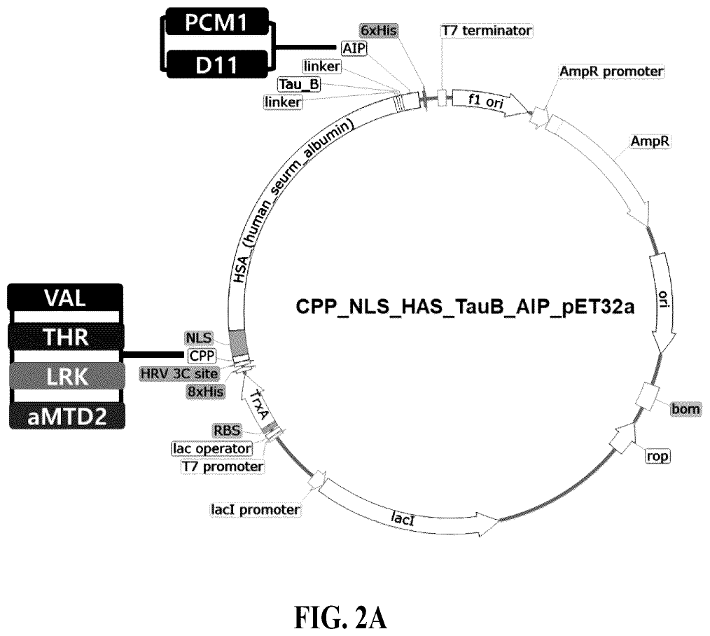

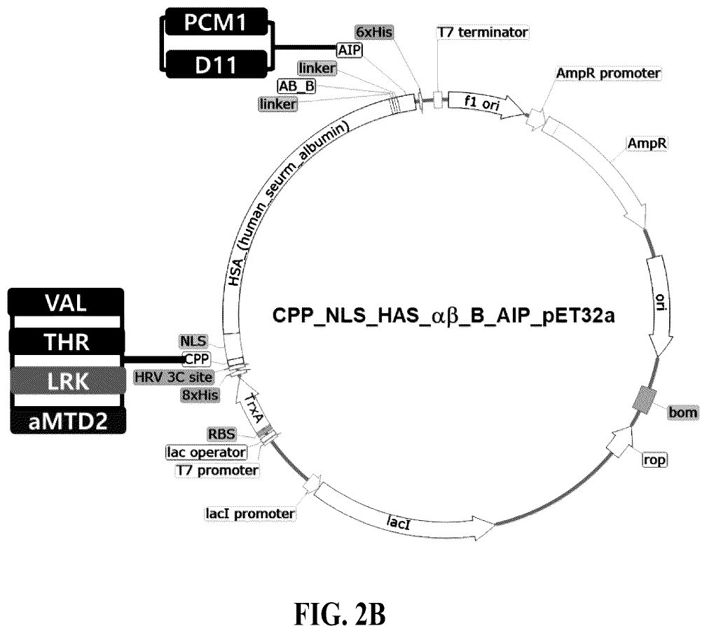

A DNA expression vector for preparing the designed fusion protein was constructed as shown in FIG. 2. Specifically, to increase cell permeability, CPP peptides VAL (SEQ ID NO: 1: VALLPAVP), THR (SEQ ID NO: 2: THRPPMWSPVWP), LRK (SEQ ID NO: 3: LRKLRKRLL), and aMTD2 (SEQ ID NO: 4: VAALLPAVVVAP, control group: US20180171322A1) were prepared. For protein stability and amplification of a HSA protein gene for penetrating the blood-brain barrier (BBB), there were used coding base sequences of an HSA protein (SEQ TD NO: 6) having an amino acid sequence from aspartate (Asp) at position 26 to leucine (Lue) at position 609, and fusion proteins having the amino acid sequences of Tau (Tau_B; SEQ TD NO: 7) and amyloid-beta (a43_B; SEQ TD NO: 8) binding peptides, and PCM1 (SEQ TD NO: 8) and D11 (SEQ TD NO: 9) peptides (FIG. 2).

To amplify each gene fragment, a PCR reaction was performed using a gene amplifier using synthesized primers. The detailed information of primers used was shown in Table 2 below.

TABLE 2

| Template | Direc- | | SEQ |
|---|---|---|---|
| DNA | tion | PrimerSequence(5′ → 3′) | ID NO: |
| Albumin_ | Forward | CCCGGTCTCCACCGGACGCCCACAAGAGCGAG | 14 |
| pET32aP | Reverse | CCCGGTCTCCGCTTTTAGAGTCCCAGAGCAACCTGA | 15 |
| VAL | Forward | GTGGCACTGCTGCCGGCGGT | 16 |
| | Reverse | CGGTACCGCCGGCAGCAGTG | 17 |
| THR | Forward | ATGACTCATCGTCCTCCTATGTGGTCTCCTGTTTGGCCT | 18 |
| | Reverse | CCATAGGCCAAACAGGAGACCACATAGGAGGACGATG | 19 |
| | | AG | |
| LRK | Forward | ATGCTTCGTAAATTACGTAAACGTTTATTA | 20 |
| | Reverse | CCATTAATAAACGTTTACGTAATTTACGAA | 21 |
| MTD2 | Forward | GTGGCGGCTCTGCTGCCTGCCGTAGTAGTAGC | 22 |
| | Reverse | CGGAGCTACTACTACGGCAGGCAGCAGAGCCG | 23 |
| THR_al- | Forward | CCCGGTCTCCACCGATGACTCATCGTCCTCCTATG | 24 |
| bumin | | | |
| LRK_al- | Forward | CCCGGTCTCCACCGATGCTTCGTAAATTACGTAAACG | 25 |
| bumin | | | |
| VAL_al- | Forward | CCCGGTCTCCACCGAGTGGCACTGCTGCCGGCGGT | 26 |
| bumin | | | |
| MTD2_ | Forward | CCCGGTCTCCACCGAGTGGCGGCTCTGCTGCCTGCCGT | 27 |
| albumin | | AGTAGTAGC | |
| NLS | Forward | CCCGGTCTCCACCGCGGAAAAGAGCTCGGGCTG | 28 |
| | Reverse | CCCGGTCTCCCGTCGTTCTCGACCCTTCTGCGT | 29 |
| Tau_B | Forward | CCGGCTCCGTGCAGATAATTAATAAGGGCA | 30 |
| | Reverse | CCACTGCCCCTTATTAATTATCTGCACGGAG | 31 |
| Aβ_B | Forward | CCGGCTCCCTGTACATCTGGATCTGGAGAACCGGCA | 32 |
| | Reverse | CCACTGCCGGTTCTCCAGATCCAGATGTACAGGGAG | 33 |
| PCM1 | Forward | CCCGGTCTCCGTGGGTCCAGTCAAAAGTCTGATGAAGA | 34 |
| | | AG | |
| | Reverse | CCCGGTCTCCGCTTTTATATTGTCAGTTTCAGTGGTAAAT | 35 |
| D11 | Forward | CCCGGTCTCCGTCCTATGGTCGTAAAAAACGTCGTC | 36 |
| | Reverse | CCCGGTCTCCGCTTTTACACCCAGTTCGCGGTGAAG | 37 |
| pFLAG_ | Forward | cccGGTCTCcGGGTGGCATCCCTGTGACCCC | 38 |
| Vector | Reverse | cccGGTCTCcCTTGTCATCGTCATCCTTGTA | 39 |

The forward and reverse primers included a base sequence (5′-GGTCTC-3′) corresponding to a BsaI restriction enzyme recognition site. PCR was performed as follows using DNA encoding VAL, THR, LRK, aMTD2, HSA, Tau_B, αβ_B, PCM1, and D11 as a template. A total of 50 μl of reaction solution was prepared by adding distilled water to a mixed solution of 0.5 μl of each template DNA at a concentration of about 100 ng/μl, 1 μl of dNTP at a 10 mM concentration, 5 μl of a 5-fold concentrated PCR buffer solution (Thermofisher, USA), 1.5 μl of dimethyl sulfoxide (DMSO) at a 100% concentration, 0.5 μl each of forward and reverse primers at a concentration of 100 pmole/μl, and 0.5 μl of Phusion DNA polymerase (2 U/μl, Thermofisher, USA). The reaction solution was preheated at 98° C. for 30 seconds using a gene amplifier, then the reaction was repeated 30 times for 10 seconds at 98° C., 30 seconds at 60° C., and 15 seconds at 72° C., and the final amplification step was performed at 72° C. for 5 minutes. The reaction solution was separated by electrophoresis on a 0.8% agarose gel, and the genes were eluted.

Each DNA fragment and a pET32a vector fragment of a dementia-inhibiting fusion protein obtained in the step were added to each reaction tube at a molecular ratio of 2:1, and then added with 2 μl of a 10-fold concentrated ligation reaction buffer, 1 μl each of T4 DNA ligase (400 U/μl, NEB, USA) and BsaI restriction enzyme (10 U/μl, NEB, USA), and added with distilled water to become a total of 20 μl. The reaction solution was reacted at 37° C. for 1 hour, and then the enzymes in the reaction tubes were inactivated at 65° C. for 5 minutes. The reaction solution was added to E. coli DH5a (Thermofisher, USA) competent cells to be transformed and then streak-plated on an LB solid medium containing 100 μg/ml ampicillin to select transformants. The plasmids were extracted from E. coli, and through sequencing analysis, respective dementia-inhibiting fusion protein DNA fragments VAL, THR, LRK, aMTD2, HSA, Tau_B, αβ_B, PCM1, and D11 were linked to the pET32a plasmid containing HAS to confirm expression vectors CPP-NLS-HAS_Tau_B_AIP_pET32a and CPP-NLS-HAS_αβ_B_AIP_pET32a.

Additionally, for expression in human cells, previously cloned CPP-NLS-HAS_Tau_B_AIP_pET32a and CPP-NLS-HAS_αβ_B_AIP_pET32 were placed in the pFLAG vector, and a PCR reaction was performed with a gene amplifier using synthesized primers to amplify each gene fragment and then performed similarly to the cloning method above.

### 2-2. Preparation of Fusion Protein Expressed Transformants

In order to prepare a fusion protein for eliminating a neurodegenerative disease-causing factor, any one of the fusion protein expressed vectors prepared in Example 2-1 was transformed in E. coli BL21(DE3)-RIL (Novagen, USA) as a protein expression host using a conventional method (Table 3).

TABLE 3

| | Expression vector |
|---|---|
| Classification | pET32aP |
| 1-1 | VAL-NLS-HSA-Tau_B-PCM1 |
| 1-2 | THR-NLS-HSA-Tau_B-PCM1 |
| 1-3 | LRK-NLS-HSA-Tau_B-PCM1 |
| 1-4 | MTD2-NLS-HSA-Tau_B-PCM1 |
| 2-1 | VAL-NLS-HSA-αβ_B-PCM1 |
| 2-2 | THR-NLS-HSA-αβ_B -PCM1 |
| 2-3 | LRK-NLS-HSA-αβ_B -PCM1 |
| 2-4 | MTD2-NLS-HSA-αβ_B -PCM1 |
| 3-1 | VAL-NLS-HSA-Tau_B-D11 |
| 3-2 | MTD2-NLS-HSA-Tau_B-D11 |
| 4-1 | VAL-NLS-HSA- αβ_B -D11 |
| 4-2 | MTD2-NLS-HSA- αβ_B -D11 |
| 5-1 | VAL-NLS-Tau_B-PCM1 |
| 5-2 | THR-NLS-Tau_B-PCM1 |
| 5-3 | LRK-NLS-Tau_B-PCM1 |
| 6-1 | VAL-NLS- αβ_B -PCM1 |
| 6-2 | THR-NLS- αβ_B -PCM1 |
| 6-3 | LRK-NLS- αβ_B -PCM1 |

### 2-3. Induction and Purification of Fusion Protein Expression

Each transformed E. coli strain was placed in 5 ml of a LB liquid medium containing 100 μg/ml of ampicillin, shaking-cultured at 37° C. for 16 hours, and then 1 ml of each was taken and subcultured in 100 ml of a LB liquid medium (100 μg/ml of ampicillin), respectively. After culturing at 37° C. until the absorbance of the culture medium was about 0.6 at a wavelength of 600 nm, the culture temperature was lowered to 18° C. and isopropyl β-D-1-thiogalactopyranoside (TPTG) was added to be a final concentration of 1 mM to induce the protein expression.

After 16 hours, 50 ml of each cell culture solution was centrifuged at 3,500 rpm for 15 minutes to collect each cell deposit. Each collected cell deposit was suspended in 1 ml of a cell lysis buffer (20 mM HEPES-hydrochloric acid buffer, pH 7.5, 1 mM Tris(2-carboxyethyl)phosphine (TCEP)) containing 150 mM or 500 mM sodium chloride, and then cells were lysed using a mini ultrasonicator (Sonifier 450, Branson, USA) in an ice bath. The lysed cell solution was centrifuged with a 4° C. centrifuge at 13,000 rpm for 10 minutes to separate the supernatant and the cell deposit, a portion of the supernatant was mixed with 20 μl of nickel (Ni) resin for 1 hour in a cold room at 4° C. and then centrifuged at 2,500 rpm for 1 minute to remove the supernatant and eliminate impurities bound to the nickel resin with the cell lysis buffer containing 20 mM imidazole.

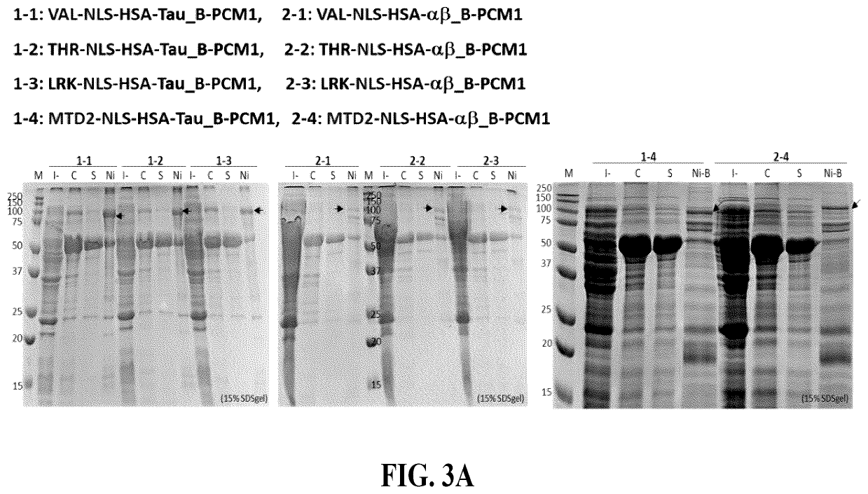

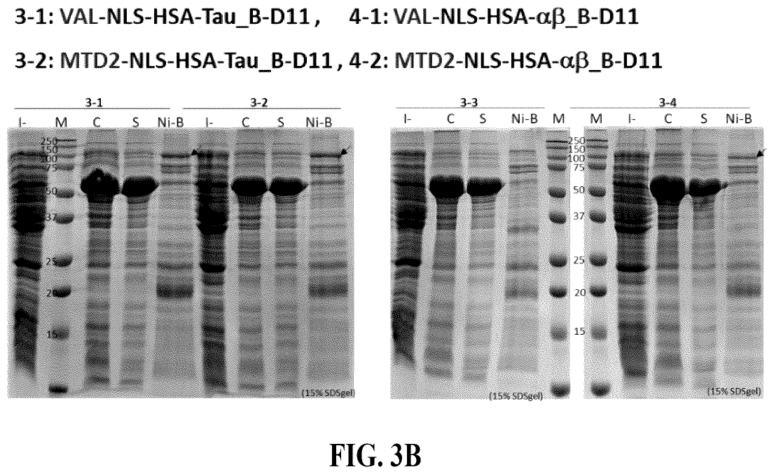

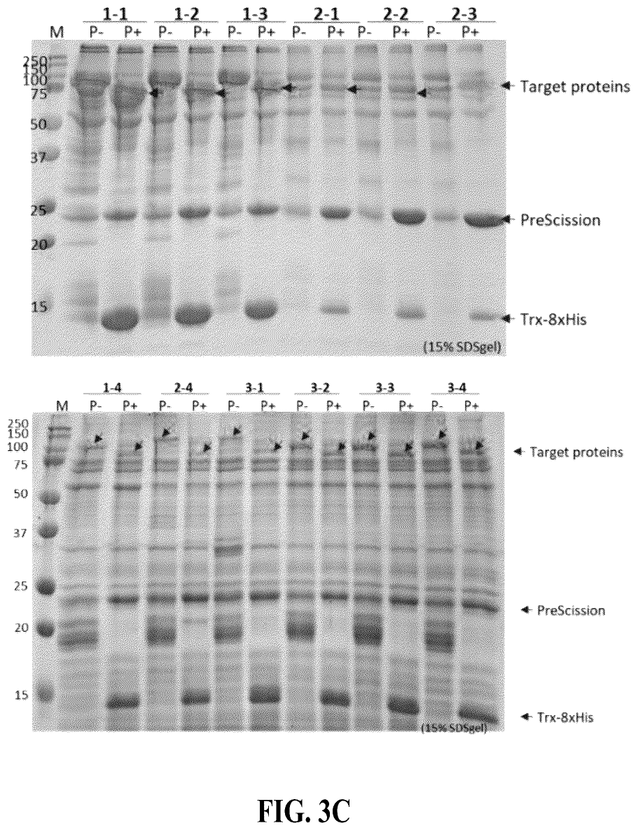

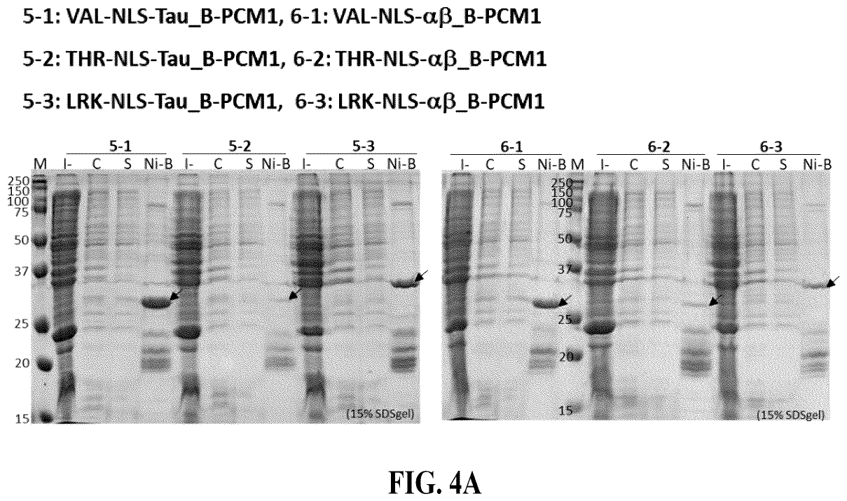

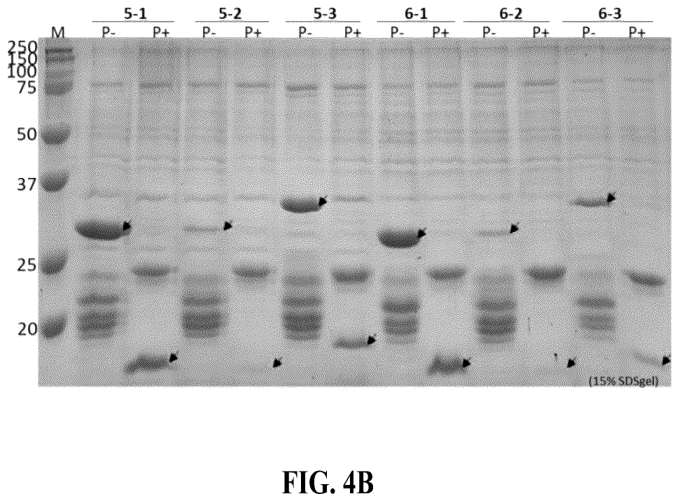

After inducing protein expression, a cell lysate, a supernatant centrifuged after cell lysis, and a sample obtained by purifying with nickel resin were used as samples (FIGS. 3 and 4).

### 2-4. Induction and Purification of Fusion Protein Expression

In order to prepare the fusion proteins designed above, specifically, the DNA plasmids encoding each of the fusion proteins designed above were transformed into E. coli BL21 cells, and then cultured overnight at 37° C. on a Luria-Bertani (LB) agar plate containing ampicillin (100 μg/ml). To induce fusion protein expression, transfected BL21 cells were cultured overnight at 18° C. in 400 ml of a LB-ampicillin medium containing 0.2 mM isopropyl β-D-1-thiogalactopyranoside (IPTG). The cells were harvested by ultracentrifugation and lysed in a lysis buffer (containing 50 mM Hepes (PH 7.4), 100 mM NaCl, 5% glycerol, 5 mM imidazole, and 1 mM phenylmethylsulfonyl fluoride (PMSF)) by sonication. After performing ultracentrifugation at 18,000 rpm at 4° C. for 40 minutes, the soluble lysate was cultured with an Ni-NTA resin (Thermo Fisher Scientific) at 4° C. for 2 hours and then purified using a poly-prep chromatography column (Bio-Rad). Column-binding proteins were eluted with a lysis buffer (containing 50 mM Hepes (PH 7.4), 50 mM NaCl, 5% glycerol, 300 mM imidazole, and 1 mM PMSF) and impurities were eliminated using an ultrafiltration spin column (Millipore). Each supernatant containing the fusion protein obtained through the above-described process passed through a nickel-nicrylotriacetic acid (Ni-NTA) column (GE Healthcare Life Sciences, USA) equilibrated with a cell lysis buffer, and then impurities were eliminated with a cell lysis buffer containing 20 mM imidazole, and complex proteins were eluted from the column using an imidazole concentration gradient from 0 to 500 mM. Thereafter, the fractions were confirmed through SDS-PAGE.

FIGS. 3 and 4 illustrate SDS-PAGE results for each of 18 types of prepared water-soluble fusion proteins for eliminating neurodegenerative disease-causing factors.

FIGS. 3 and 4 illustrate SDS-PAGE results of each water-soluble fusion proteins for eliminating neurodegenerative disease-causing factors prepared in Example 2-3 after cleaving eight histidine tags using PreScission protease.

In FIGS. 3 and 4, “M” refers to a protein marker (kDa), “P−” refers to a water-soluble fusion protein not treated with PreScission protease, and “P+” refers to a water-soluble fusion protein treated with PreScission protease (GE Healthcare Life Sciences, USA). Specifically, FIGS. 3 and 4 illustrate results measured by SDS-PAGE after reacting at 4° C. for 16 hours by adding PreScission protease (treating 2 units of PreScission protease per 0.5 mg of protein) in order to eliminate the eight histidine tags bound to the N terminus of each fusion protein prepared from Examples 2-3 and 2-4. It may be confirmed that water-soluble fusion proteins for eliminating neurodegenerative disease-causing factors may be recovered with high purity through PreScission protease.

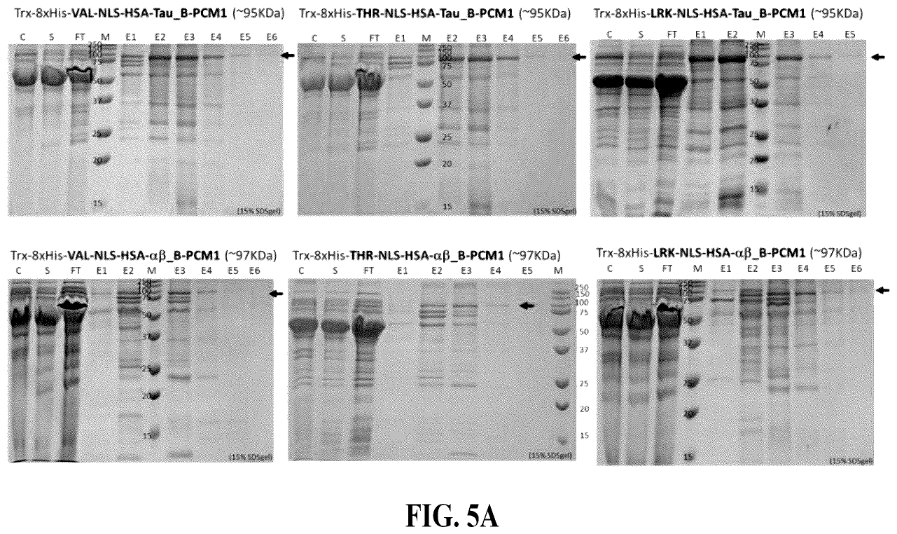

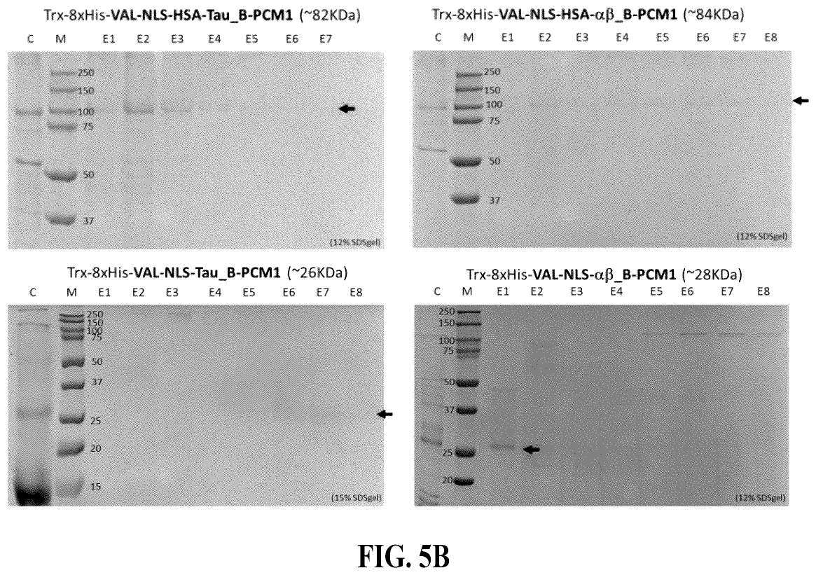

FIG. 5 illustrates the results analyzed by SDS-PAGE after separating and purifying 10 types of fusion proteins from which the histidine tags were eliminated using PreScission protease through protein exclusion chromatography. “M” refers to a protein marker (kDa), “C” refers to a fusion protein solution for eliminating each water-soluble neurodegenerative disease-causing factor before injection into protein exclusion chromatography, “S” refers to a fusion protein solution for eliminating a neurodegenerative disease-causing factor in an aqueous buffer solution, “FT” refers a flow through, and “E1” to “E8” are each fraction separated and purified by protein exclusion chromatography. Specifically, each solution containing 10 types of water-soluble fusion proteins from which the histidine tags have been eliminated was concentrated to 5 ml, and then injected into protein exclusion chromatography (Superdex 200, 26/60, GE Healthcare Life Sciences, USA) equilibrated with a buffer solution containing 150 mM sodium chloride, 20 mM HEPES-hydrochloric acid buffer, pH 7.4, and 1 mM DTT, and fractioned according to a molecular weight using a buffer solution (containing 150 mM sodium chloride, 20 mM HEPES-hydrochloric acid buffer, pH 7.4, 1 mM DTT), respectively. Each of the fractions was measured by SDS-PAGE. It may be confirmed that water-soluble fusion proteins for eliminating neurodegenerative disease-causing factors were separated, purified, and recovered with high purity (FIG. 5).

### Example 3. Confirmation of Binding to Target and Autophagy Induction of Fusion Protein Containing D11 Autophagy Inducing Peptide

### 3-1. Preparation and Transfection of Plasmids for Expressing Fusion Proteins

A polynucleotide encoding VAL-NLS-HSA-Tau_B-PCM1, VAL-NLS-Tau_B-PCM1, VAL-NLS-HSA-PCM1, VAL-NLS-HSA-αβ_B-PCM1, VAL-NLS-αβ_B-PCM1, MTD2-NLS-HAS-Tau_B-D11, or VAL-NLS-HSA-Tau_B-D11 was inserted into a pFlag-CMV™2 vector. To transfect each plasmid into HEK293T cells, 2 M CaCl₂ and 2×HBS buffer (50 mM HEPES, 10 mM KCl, 12 mM glucose, 280 mM NaCl, 1.5 mM Na₂HPO₄, pH 7.05) were used, and to transfect each plasmid into HeLa cells and A549 cells, an Effectene (Qiagen, USA) reagent was used according to the manufacturer's manual.

### 3-2. Western Blot Assay

Each cell was obtained, and proteins were prepared using a protein lysis buffer (50 mM Tris-Cl, 150 mM NaCl, 0.5% NP40, 1 mM EDTA, pH 7.5). 20 to 50 μg of proteins were boiled with a 6×SDS sample buffer at 100° C. for 5 minutes and then separated by SDS-PAGE on a 6 to 15% gel. The proteins in the gel were transferred to a nitrocellulose membrane (Pall Corporation, USA), blocked using 5% non-fat milk, and incubated with primary antibodies at 4° C. overnight. Thereafter, the membrane was incubated with secondary antibodies for 1 hour at room temperature, reacted with an ECL solution (Santa Cruz Biotechnology, USA), and visualized using ChemiDoc (ATTO Technology, USA). The primary antibodies used in the experiment were as follows: Primary antibodies, rabbit anti-p62 and rabbit anti-LC3B (Cell signaling, USA), mouse anti-Flag (Sigma-Aldrich, USA), rabbit anti-Tau (abcam, USA), rabbit anti-β-actin (Abfrontier, Korea), rabbit anti-Beclin1 and mouse anti-HSP90α/β (Santa Cruz Biotechnology, USA); Secondary antibodies, Goat anti-Rabbit and Goat anti-Mouse (GenDEPOT, USA).

### 3-3. Immunoprecipitation

For an experiment to confirm the binding between proteins, HEK293T cells were transfected with a Tau expression vector containing GFP (GFP-Tau) and vectors containing the previously prepared Flag peptide, and the cells were obtained 24 hours later. Thereafter, 3 to 5 mg of proteins were prepared using a protein lysis buffer, added with anti-Flag agarose beads (Sigma-Aldrich, USA), and incubated at 4° C. for 4 to 6 hours. After washing, the remaining beads were added with a 6×sample buffer and boiled at 100° C. for 10 minutes. Each sample was visualized through Western blot.

### 3-4. Confirmation of Binding of Fusion Protein to Tau Protein

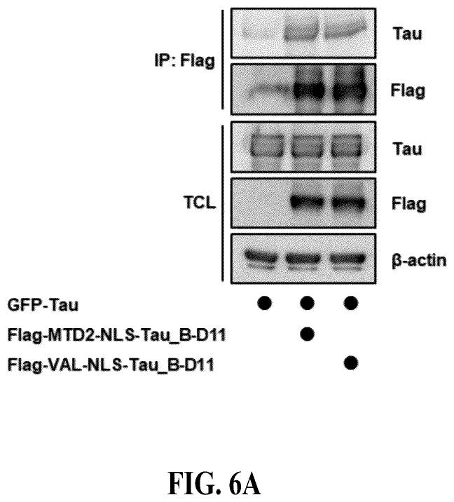

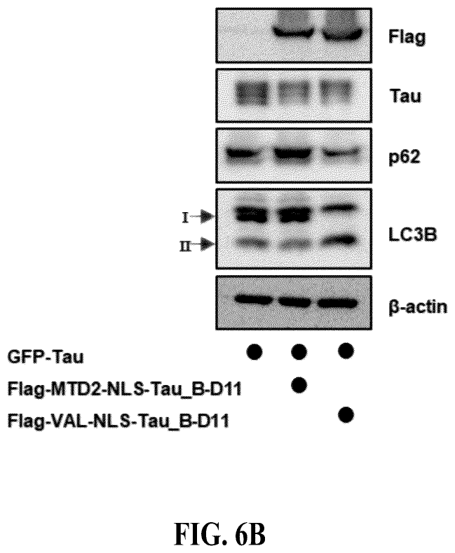

To confirm whether the MTD2-NLS-HSA-Tau_B-D11 and VAL-NLS-HSA-Tau_B-D11 fusion proteins newly combined by the present inventors bound to the Tau protein, plasmids MTD2-NLS-HSA-Tau_B-D11, VAL-NLS-HSA-Tau_B-D11 and GFP-Tau were transfected into HEK293T cells and then tested using immunoprecipitation, respectively. As a result, it was confirmed that both fusion proteins bound to the Tau protein (FIG. 6A).

### 3-5. Confirmation of Autophagy Induction of Fusion Protein

Next, in order to determine whether a D11 sequence contained in both fusion proteins induced intracellular autophagy activation to reduce the amount of Tau protein aggregated by a Tau_B peptide, GFP-Tau plasmid and MTD2-NLS-HSA-Tau_B-D11 and VAL-NLS-HSA-Tau_B-D11 plasmids were transfected into HeLa cells for 24 hours and then subjected to Western blot assay. As a result of confirming the expression of p62 protein, a representative autophagy marker, the amount thereof was decreased by MTD2-NLS-HSA-Tau_B-D11 and VAL-NLS-HSA-Tau_B-D11, and the amount of aggregated Tau protein was also decreased compared to a control group. This means that the autophagy was activated by MTD2-NLS-HSA-Tau_B-D11 or VAL-NLS-HSA-Tau_B-D11, and the Tau protein bound to MTD2-NLS-HSA-Tau_B-D11 and VAL-NLS-HSA-Tau_B-D11 was degraded by autophagy activation. However, the conversion of LC3B-I to LC3B-II, as another important marker of autophagy activity, was increased compared to the control group only in cells expressing VAL-NLS-HSA-Tau_B-D11 (FIG. 6B). Therefore, in subsequent experiments, a newly combined plasmid using VAL peptide was used.

### Example 4. Confirmation of Binding to Target and Autophagy Induction of Fusion Protein Containing PCM1 Autophagy Inducing Peptide

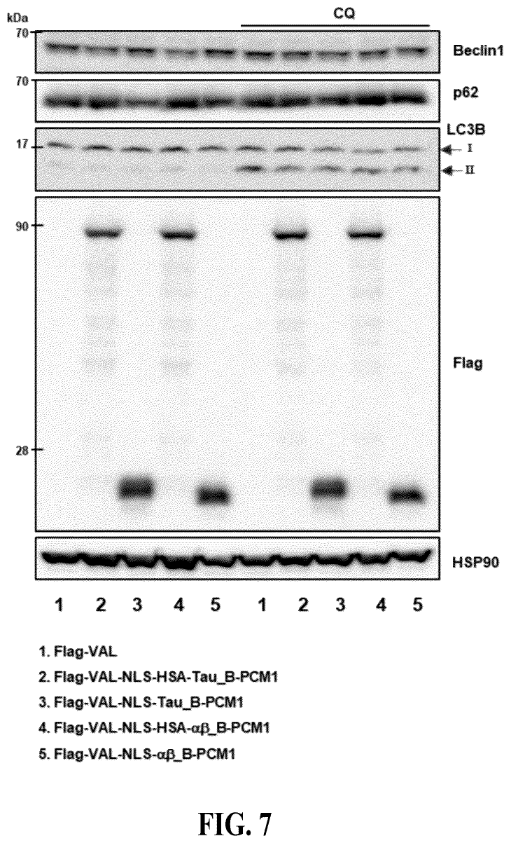

New plasmids VAL-NLS-HSA-Tau_B-PCM1 and VAL-NLS-HSA-αβ_B-PCM1 were prepared by combining a PCM1 sequence instead of the D11 sequence of the AIP peptide. To confirm whether the PCM1 sequence may induce autophagy activation, the two newly prepared plasmids were each transfected into A549 cells to confirm the amount of autophagy marker protein. As a result, the two fusion proteins induced the activation of autophagy (FIG. 7). However, in an experimental group treated with an autophagy inhibitor chloroquine (CQ), there were no changes in autophagy marker proteins by the two fusion proteins (FIG. 7).

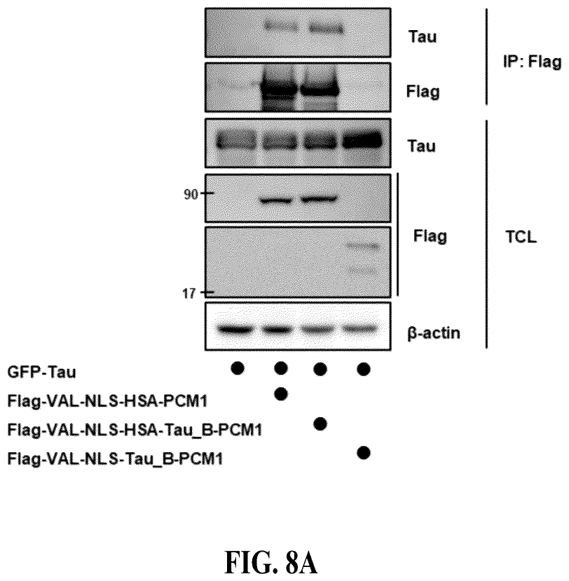

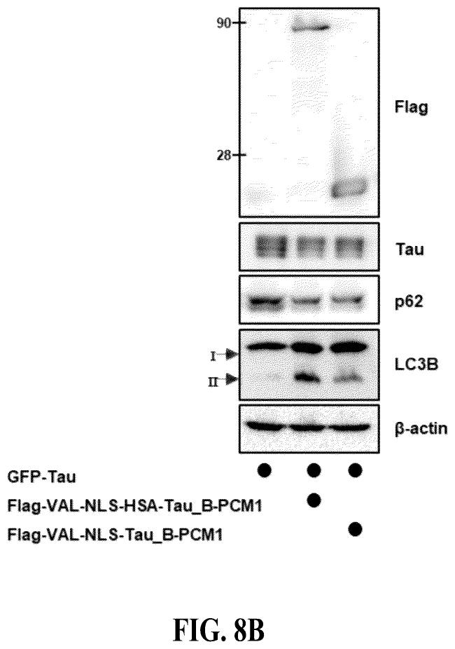

Lastly, it was confirmed whether the newly combined fusion proteins could eliminate the Tau protein, a neurodegenerative disease-causing factor. First, to confirm the binding between the Tau protein and the fusion protein, GFP-Tau and VAL-NLS-HSA-Tau_B-PCM1 were overexpressed in HEK293T cells. As a result, it was confirmed that the fusion protein and the Tau protein were bound (FIG. 8A). Thereafter, to confirm whether the binding was caused by the Tau_B peptide or human serum albumin (HSA) protein, VAL-NLS-HSA-PCM1 without GFP-Tau and Tau_B peptides and VAL-NLS-Tau_B-PCM1 without HSA protein were each overexpressed in HEK293T cells to confirm binding to the Tau protein. As a result, only the fusion proteins VAL-NLS-HSA-Tau_B-PCM1 and VAL-NLS-HSA-PCM1 containing the HSA protein bound to the Tau protein (FIG. 8A). This suggests that the HSA domain is involved in binding of the fusion protein to the Tau protein together with the Tau-B peptide.

Next, GFP-Tau plasmid, and VAL-NLS-HSA-Tau_B-PCM1 and VAL-NLS-Tau_B-PCM1 plasmids were transfected into HeLa cells for 24 hours, and then Western blot assay was performed. In both samples containing VAL-NLS-HSA-Tau_B-PCM1 and VAL-NLS-Tau_B-PCM1, the amount of p62 protein was decreased compared to the control group, and the conversion of LC3B-I to LC3B-II was increased, and at this time, the amount of aggregated Tau protein was decreased (FIG. 8B).

This shows that the fusion protein designed in the present disclosure may eliminate neurodegenerative disease-causing factors by inducing autophagy activation.

### Example 5. Confirmation of Effects of Fusion Proteins in Tau Overexpressed Dementia Model

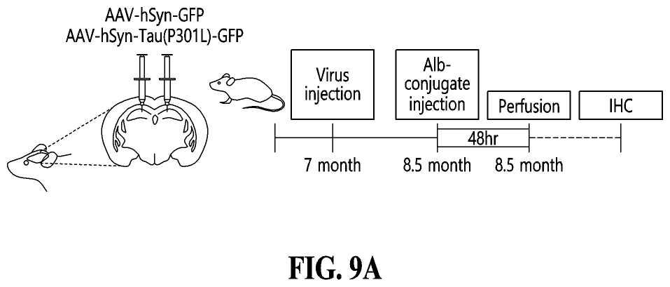

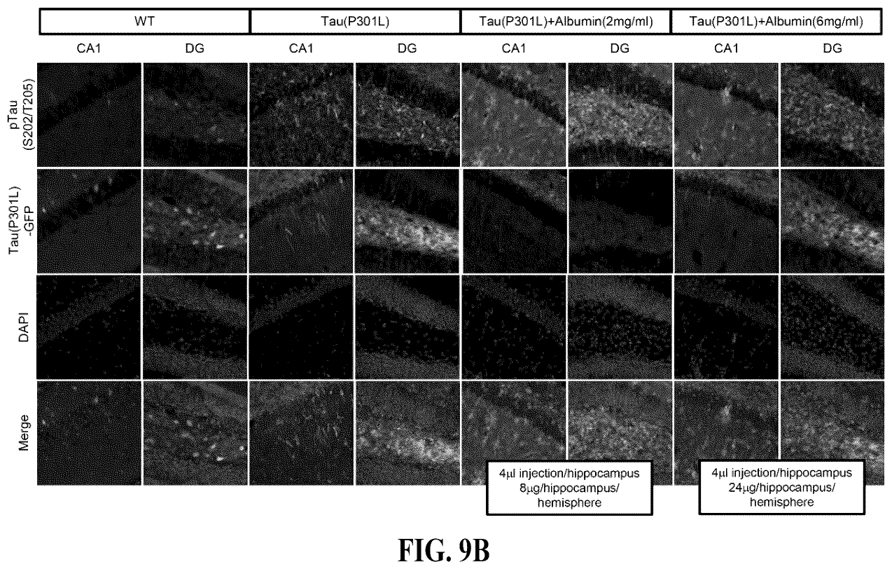

A schematic diagram of the experiment is shown in FIG. 9A. A 7-month-old mouse was anesthetized with avertine and placed in stereotaxic frames (Stoelting Co.), and then the scalp was incised, and two holes were drilled in the skull above the two hippocampi. A total of 2.5 μl AAV-hSyn-GFP or AAV-hSyn-Tau (P301L) virus was injected into the bilateral hippocampi of the mouse (AP: −2.0 mm, ML: +/−1.25 mm, DV: −1.4/−2.0 mm) at a rate of 0.5 μl/min using a syringe pump. Six weeks after virus injection, the fusion protein VAL-NLS-HSA-Tau_B-PCM1 was directly injected in the dentate gyrus (DG) in the hippocampus area at low (8 mg/hippocampus/hemisphere) or high concentration (24 mg/hippocampus/hemisphere) depending on an experimental group. 48 hours after the injection of the albumin-bound scavenger, the mouse was perfused with 4% paraformaldehyde (PFA), and neuropathological and biochemical analysis were performed. Phosphorylated Tau (pTau) was immunostained with an AT8 monoclonal antibody specific for pTau in S202/T205 and observed under a microscope.

As a result, the level of total Tau-GFP signal (green) was reduced in the dentate gyrus (DG) of the hippocampus of low- and high-dose administered mouse. As a result, it may be seen that the fusion protein of the present disclosure induces the degradation of Tau protein in nerve cells. Meanwhile, in the low-dose group, there was no effect on the level of pTau (S202/T205) signal (red), whereas in the high-dose group, the level of pTau (S202/T205) signal (red) confirmed in the DG of the hippocampus of Tau (P301L)-GFP mouse has decreased. In other words, it may be seen that pTau (S202/T205), which is toxic to nerve cells and causes dementia, is eliminated by administering a high concentration of fusion protein, but the effect is poor at a low concentration (FIG. 9B).

As described above, although the examples have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other examples, and equivalents to the appended claims fall within the scope of the claims to be described below.

The present disclosure was completed with the support of the Korea Institute of Science and Technology as follows:
1. Account number: 2V09350, Project Title: PRIDE up based technology utilizing support project
2. Account number: 2V09740, Project Title: Study on common technology based construction and technology support
3. Account number: 2V0935C, Project Title: Study on activity regulation mechanism and structure identification of deubiquitination enzyme USP35
4. Account number: 2V10051, Project Title: Study on common technology based construction and technology support

What is claimed is:

1. A fusion protein for eliminating a neurodegenerative disease-causing factor, wherein
the neurodegenerative disease-causing factor is Tau or amyloid-beta, and
the following peptides (1) to (4) are linked in order from an N-terminus to a C-terminus of the fusion protein:
(1) a cell penetrating peptide (CPP)
(2) a human serum albumin-derived peptide (HSA)
(3) a neurodegenerative disease-causing factor binding peptide
(4) an autophagy inducing peptide (AIP).

2. The fusion protein of claim 1, wherein the (1) CPP comprises one or more amino acid sequences selected from the group consisting of SEQ ID NOs: 1 to 3.

3. The fusion protein of claim 1, wherein the (2) HAS comprises an amino acid sequence represented by SEQ ID NO: 6.

4. The fusion protein of claim 1, wherein the (3) neurodegenerative disease-causing factor binding peptide comprises an amino acid sequence represented by SEQ ID NO: 7 or 8.

5. The fusion protein of claim 1, wherein the (4) AIP comprises an amino acid sequence represented by SEQ ID NO: 9 or 10.

6. The fusion protein of claim 1, wherein the (1) CPP and the (2) HSA are linked by a nuclear localization signal (NLS) peptide.

7. The fusion protein of claim 6, wherein the NLS peptide comprises an amino acid sequence represented by SEQ ID NO: 5.

8. The fusion protein of claim 1, wherein the (2) HSA and the (3) neurodegenerative disease-causing factor binding peptide are linked by a linker.

9. The fusion protein of claim 1, wherein the (3) neurodegenerative disease-causing factor binding peptide and the (4) AIP are linked by a linker.

10. The fusion protein of claim 8, wherein the linker comprises an amino acid sequence represented by SEQ ID NO: 40.

11. A polynucleotide encoding the fusion protein of claim 1.

12. A pharmaceutical composition for preventing or treating degenerative brain diseases comprising the fusion protein of claim 1 as an active ingredient.

13. The pharmaceutical composition of claim 12, wherein the degenerative brain diseases are one or more diseases selected from the group consisting of dementia, Alzheimer's disease, Lou Gehrig's disease, Lewy dementia, frontotemporal dementia, Parkinson's disease, tremor, proximal lateral sclerosis, chorea, multiple sclerosis, progressive supranuclear palsy, and Huntington's disease.


SEQUENCE LISTING (41 sequences)

SEQ ID NO: 1 - protein, 8 aa, synthetic construct
  REGION 1..8: note = VAL
VALLPAVP

SEQ ID NO: 2 - protein, 12 aa, synthetic construct
  source 1..12: note = THR
THRPPMWSPVWP

SEQ ID NO: 3 - protein, 9 aa, synthetic construct
  source 1..9: note = LRK
LRKLRKRLL

SEQ ID NO: 4 - protein, 12 aa, synthetic construct
  source 1..12: note = aMTD2
VAALLPAVVVAP

SEQ ID NO: 5 - protein, 49 aa, synthetic construct
  REGION 1..49: note = NLS
RKRARAEKKALKKKKKIRGPEKRGADEDDEKEWGDDEEEQPSKRRRVEN

SEQ ID NO: 6 - protein, 585 aa, synthetic construct
  source 1..585: note = HSA
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQVALGL

SEQ ID NO: 7 - protein, 6 aa, synthetic construct
  source 1..6: note = Tau_B
VQIINK

SEQ ID NO: 8 - protein, 8 aa, synthetic construct
  REGION 1..8: note = alphabeta_B
LYIWIWRT

SEQ ID NO: 9 - protein, 21 aa, synthetic construct
  source 1..21: note = PCM1
SQKSDEEDFVKVEDLPLKLTI

SEQ ID NO: 10 - protein, 24 aa, synthetic construct
  source 1..24: note = D11
YGRKKRRQRRRGGDHWIHFTANWV

SEQ ID NO: 11 - DNA, 1779 bp, synthetic construct
  misc_feature 1..1779: note = humanserumalbumingene
  source 1..1779: mol_type = other DNA
gaggcacataaaagcgaagttgcacaccgtttcaaagacctgggcgaagaaaatttcaaagcactggttgaaaatctgtatttccaaggcctgattgcctttgcacagtatctgcaacaatgcccgttcgaagatcatgtcaaactggtgaacgaagttaccgaatttgccaaaacgtgcgtcgcagatgaatcagctgaaaattgtgacaaatcgctgcacaccctgtttggcgacaaactgtgcaccgtggcaacgctgcgtgaaacgtacggtgaaatggcggattgctgtgccaaacaggaaccggaacgcaacgaatgctttctgcaacataaagatgacaatccgaatctgccgcgtctggtgcgtccggaagttgatgtcatgtgtaccgcatttcatgacaatgaagaaacgttcctgaaaaaatatctgtacgaaatcgcgcgtcgccacccgtatttttacgcgccggaactgctgtttttcgccaaacgttataaagcggccttcaccgaatgctgtcaggcagctgataaagcggcctgcctgctgccgaaactggatgaactgcgtgacgaaggcaaagcgagcagcgccaaacagcgcctgaaatgtgcatctctgcaaaaatttggtgaacgtgctttcaaagcgtgggccgttgcacgtctgtctcagcgttttccgaaagcagaatttgctgaagtgagcaaactggttaccgatctgacgaaagtgcataccgaatgctgtcacggcgatctgctggaatgtgccgatgaccgtgcggacctggccaaatatatttgtgaaaaccaggattcgatcagttccaaactgaaagaatgctgtgaaaaaccgctgctggaaaaatctcattgcattgccgaagttgaaaacgatgaaatgccggcagacctgccgagcctggcagctgatttcgttgaatctaaagacgtctgtaaaaattatgcagaagctaaagacgtgtttctgggcatgttcctgtatgaatacgcgcgtcgccatccggattatagcgtggttctgctgctgcgtctggccaaaacctacgaaaccacgctggaaaaatgctgtgcggcggccgatccgcatgaatgctacgcgaaagtctttgatgaatttaaaccgctggttgaagaaccgcagaacctgatcaaacaaaattgtgaactgtttgaacagctgggtgaatataaattccaaaacgccctgctggttcgctacaccaagaaagtgccgcaggtgagtaccccgaccctggtggaagtgagccgtaatctgggcaaagtgggttcaaaatgctgtaaacatccggaagcaaaacgtatgccgtgcgctgaagattatctgtccgtcgtgctgaatcaactgtgtgtgctgcacgaaaaaaccccggtctcagatcgtgtgaccaaatgctgtacggaatcgctggttaaccgtcgcccgtgctttagcgcgctggaagtggatgaaacgtacgttccgaaagaatttaatgcagaaacctttacgttccatgctgatatttgtaccctgagcgaaaaagaacgccagatcaaaaaacaaacggcgctggttgaactggtcaaacacaaaccgaaagcgaccaaagaacagctgaaagccgttatggatgactttgctgcgttcgtcgaaaaatgctgtaaagccgatgacaaagaaacgtgtttcgcggaagaaggcaaaaaactggtcgcagcatcgcaggcggctctgggtctgtaa

SEQ ID NO: 12 - protein, 441 aa, synthetic construct
  source 1..441: note = Taupeptide
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 13 - protein, 2024 aa, synthetic construct
  REGION 1..2024: note = PCM1
MATGGGPFEDGMNDQDLPNWSNENVDDRLNNMDWGAQQKKANRSSEKNKKKFGVESDKRVTNDISPESSPGVGRRRTKTPHTFPHSRYMSQMSVPEQAELEKLKQRINFSDLDQRSIGSDSQGRATAANNKRQLSENRKPFNFLPMQINTNKSKDASTNPPNRETIGSAQCKELFASALSNDLLQNCQVSEEDGRGEPAMESSQIVSRLVQIRDYITKASSMREDLVEKNERSANVERLTHLIDHLKEQEKSYMKFLKKILARDPQQEPMEEIENLKKQHDLLKRMLQQQEQLRALQGRQAALLALQHKAEQAIAVMDDSVVAETAGSLSGVSITSELNEELNDLIQRFHNQLRDSQPPAVPDNRRQAESLSLTREVSQSRKPSASERLPDEKVELFSKMRVLQEKKQKMDKLLGELHTLRDQHLNNSSASPQRSVDQRSTSAPSASVGLAPVVNGESNSLTSSVPYPTASLVSQNESENEGHLNPSEKLQKLNEVRKRLNELRELVHYYEQTSDMMTDAVNENRKDEETEESEYDSEHENSEPVTNIRNPQVASTWNEVNSHSNAQCVSNNRDGRTVNSNCEINNRSAANIRALNMPPSLDCRYNREGEQEIHVAQGEDDEEEEEEAEEEGVSGASLSSHRSSLVDEHPEDAEFEQKINRLMAAKQKLRQLQDLVAMVQDDDAAQGVISASASNLDDFYPAEEDTKQNSNNTRGNANKTQKDTGVNEKAREKFYEAKLQQQQRELKQLQEERKKLIDIQEKIQALQTACPDLQLSAASVGNCPTKKYMPAVTSTPTVNQHETSTSKSVFEPEDSSIVDNELWSEMRRHEMLREELRQRRKQLEALMAEHQRRQGLAETASPVAVSLRSDGSENLCTPQQSRTEKTMATWGGSTQCALDEEGDEDGYLSEGIVRTDEEEEEEQDASSNDNFSVCPSNSVNHNSYNGKETKNRWKNNCPFSADENYRPLAKTRQQNISMQRQENLRWVSELSYVEEKEQWQEQINQLKKQLDFSVSICQTLMQDQQTLSCLLQTLLTGPYSVMPSNVASPQVHFIMHQLNQCYTQLTWQQNNVQRLKQMLNELMRQQNQHPEKPGGKERGSSASHPPSPSLFCPFSFPTQPVNLFNIPGFTNFSSFAPGMNFSPLFPSNFGDFSQNISTPSEQQQPLAQNSSGKTEYMAFPKPFESSSSIGAEKPRNKKLPEEEVESSRTPWLYEQEGEVEKPFIKTGFSVSVEKSTSSNRKNQLDTNGRRRQFDEESLESFSSMPDPVDPTTVTKTFKTRKASAQASLASKDKTPKSKSKKRNSTQLKSRVKNIRYESASMSSTCEPCKSRNRHSAQTEEPVQAKVFSRKNHEQLEKIIKCNRSTEISSETGSDFSMFEALRDTIYSEVATLISQNESRPHFLIELFHELQLLNTDYLRQRALYALQDIVSRHISESHEKGENVKSVNSGTWIASNSELTPSESLATTDDETFEKNFERETHKISEQNDADNASVLSVSSNFEPFATDDLGNTVIHLDQALARMREYERMKTEAESNSNMRCTCRIIEDGDGAGAGTTVNNLEETPVIENRSSQQPVSEVSTIPCPRIDTQQLDRQIKAIMKEVIPFLKEHMDEVCSSQLLTSVRRMVLTLTQQNDESKEFVKFFHKQLGSILQDSLAKFAGRKLKDCGEDLLVEISEVLFNELAFFKLMQDLDNNSITVKQRCKRKIEATGVIQSCAKEAKRILEDHGSPAGEIDDEDKDKDETETVKQTQTSEVYDGPKNVRSDISDQEEDEESEGCPVSINLSKAETQALTNYGSGEDENEDEEMEEFEEGPVDVQTSLQANTEATEENEHDEQVLQRDFKKTAESKNVPLEREATSKNDQNNCPVKPCYLNILEDEQPLNSAAHKESPPTVDSTQQPNPLPLRLPEMEPLVPRVKEVKSAQETPESSLAGSPDTESPVLVNDYEAESGNISQKSDEEDFVKVEDLPLKLTIYSEADLRKKMVEEEQKNHLSGEICEMQTEELAGNSETLKEPETVGAQSI

SEQ ID NO: 14 - DNA, 32 bp, synthetic construct
  misc_feature 1..32: note = Albumin_pET32aP_Primer_F
  source 1..32: mol_type = other DNA
cccggtctccaccggacgcccacaagagcgag

SEQ ID NO: 15 - DNA, 36 bp, synthetic construct
  misc_feature 1..36: note = Albumin_pET32aP_Primer_R
  source 1..36: mol_type = other DNA
cccggtctccgcttttagagtcccagagcaacctga

SEQ ID NO: 16 - DNA, 20 bp, synthetic construct
  misc_feature 1..20: note = VAL_Primer_F
  source 1..20: mol_type = other DNA
gtggcactgctgccggcggt

SEQ ID NO: 17 - DNA, 20 bp, synthetic construct
  misc_feature 1..20: note = VAL_Primer_R
  source 1..20: mol_type = other DNA
cggtaccgccggcagcagtg

SEQ ID NO: 18 - DNA, 39 bp, synthetic construct
  misc_feature 1..39: note = THR_Primer_F
  source 1..39: mol_type = other DNA
atgactcatcgtcctcctatgtggtctcctgtttggcct

SEQ ID NO: 19 - DNA, 39 bp, synthetic construct
  misc_feature 1..39: note = THR_Primer_R
  source 1..39: mol_type = other DNA
ccataggccaaacaggagaccacataggaggacgatgag

SEQ ID NO: 20 - DNA, 30 bp, synthetic construct
  misc_feature 1..30: note = LRK_Primer_F
  source 1..30: mol_type = other DNA
atgcttcgtaaattacgtaaacgtttatta

SEQ ID NO: 21 - DNA, 30 bp, synthetic construct
  misc_feature 1..30: note = LRK_Primer_R
  source 1..30: mol_type = other DNA
ccattaataaacgtttacgtaatttacgaa

SEQ ID NO: 22 - DNA, 32 bp, synthetic construct
  misc_feature 1..32: note = MTD2_Primer_F
  source 1..32: mol_type = other DNA
gtggcggctctgctgcctgccgtagtagtagc

SEQ ID NO: 23 - DNA, 32 bp, synthetic construct
  misc_feature 1..32: note = MTD2_Primer_R
  source 1..32: mol_type = other DNA
cggagctactactacggcaggcagcagagccg

SEQ ID NO: 24 - DNA, 35 bp, synthetic construct
  misc_feature 1..35: note = THR_albumin_Primer_F
  source 1..35: mol_type = other DNA
cccggtctccaccgatgactcatcgtcctcctatg

SEQ ID NO: 25 - DNA, 37 bp, synthetic construct
  misc_feature 1..37: note = LRK_albumin_Primer_F
  source 1..37: mol_type = other DNA
cccggtctccaccgatgcttcgtaaattacgtaaacg

SEQ ID NO: 26 - DNA, 35 bp, synthetic construct
  misc_feature 1..35: note = VAL_albumin_Primer_F
  source 1..35: mol_type = other DNA
cccggtctccaccgagtggcactgctgccggcggt

SEQ ID NO: 27 - DNA, 47 bp, synthetic construct
  misc_feature 1..47: note = MTD2_albumin_Primer_F
  source 1..47: mol_type = other DNA
cccggtctccaccgagtggcggctctgctgcctgccgtagtagtagc

SEQ ID NO: 28 - DNA, 33 bp, synthetic construct
  misc_feature 1..33: note = NLS_Primer_F
  source 1..33: mol_type = other DNA
cccggtctccaccgcggaaaagagctcgggctg

SEQ ID NO: 29 - DNA, 33 bp, synthetic construct
  misc_feature 1..33: note = NLS_Primer_R
  source 1..33: mol_type = other DNA
cccggtctcccgtcgttctcgacccttctgcgt

SEQ ID NO: 30 - DNA, 30 bp, synthetic construct
  misc_feature 1..30: note = Tau_B_Primer_F
  source 1..30: mol_type = other DNA
ccggctccgtgcagataattaataagggca

SEQ ID NO: 31-41 - skipped (11 entries)